(19) United States
(12) Patent Application Publication
(10) Pub. No.: US 2001/0032339 A1
(43) Pub. Date: Oct. 18, 2001

(54) Chaperone suppression of ataxin-1 aggregation and altered subcellular proteasome localization imply protein misfolding in SCA1

(76) Inventors: Huda Y. Zoghbi, Houston, TX (US); Harry T. Orr, Minneapolis, MN (US); Christopher J. Cummings, San Francisco, CA (US)

Correspondence Address: FULBRIGHT & JAWORSKI, LLP, 1301 MCKINNEY, SUITE 5100, HOUSTON, TX, 77010-3095, US

(21) Appl. No.: 09/884,384

(22) Filed: Jun. 19, 2001

Related U.S. Application Data
(62) Division of application No. 09/321,916, filed on May 28, 1999.
(60) Provisional application No. 60/087,128, filed on May 29, 1998.

(51) Int. Cl. A01K 67/00; A01K 67/027

(52) U.S. Cl. 800/13; 800/18

(57) ABSTRACT

The present invention provides a novel method for treating neurodegenerative disease in mammals. This method involves the introduction of a therapeutic effective amount of a chaperone, a chaperone-like-compound or a compound which increases proteasome activity into the neurological system of the mammal. There is also a novel method for screening for compounds having chaperone-like activity or having activity to increase proteasome activity. The screening works in either cultured cells or animal models.

### BACKGROUND OF THE INVENTION

1. Field of the Invention

The present invention relates generally to the field of chaperones and proteasomes. More particularly it relates to the use of chaperones and/or proteasome modulators for the treatment of neurodegenerative disorders and for the screening of compounds which effectively act as chaperones and/or enhance activity of proteasomes and are used for the treatment of neurodegenerative disorders.

2. Description of the Related Art

The presence of insoluble aggregates is a hallmark of a growing number of neurodegenerative disorders such as Alzheimer disease, Parkinson disease, the prion disorders, Huntington disease (HD), dentatorubral-pallidoluysian atrophy (DRPLA) and spinocerebellar ataxia type 1 and 3 (SCA1 and SCA3)¹⁻⁸. The latter four are members of a subcategory of disorders caused by a polyglutamine tract expansion. SCA1 is characterized by ataxia, progressive motor deterioration and loss of cerebellar Purkinje cells and brainstem neurons⁹. It has recently been demonstrated that mutant ataxin-1 localizes to subnuclear aggregates in COS cells, cerebellar Purkinje cells of transgenic mice, and brain stem neurons in SCA1 patients⁷. Studies of HD patients, transgenic mice, and SCA3 patients have also revealed the presence of nuclear inclusions in affected neurons^{4,5,7,8}. The mechanism that leads to protein aggregation is unknown, but one possibility is that the normal protein conformation is destabilized by the presence of the expanded polyglutamine tract, which in turn leads to abnormal protein-protein interactions and perhaps the formation of β-sheet structures^{10,11}. Over time, the accumulation of this misfolded protein could result in pathological, insoluble nuclear aggregates which perturb the nuclear function of affected neurons⁷.

Molecular chaperones might be involved in the actual formation of nuclear aggregates by stabilizing the unfolded protein in an intermediate conformation which has the propensity to interact with neighboring, unfolded proteins³³⁻³⁵. The yeast chaperone Hsp104 was shown to be necessary, at intermediate levels, for the propagation of the prion-like factor [PSI+], but when Hsp104 was overexpressed [PSI+] was lost³⁶. Thus, in yeast, it is possible to upregulate or modulate the levels of molecular chaperones to abate aggregate formation^{33, 34}.

The finding that the nuclear aggregates in SCA1 are ubiquitin-positive raised the possibility that the proteolytic pathway in these cells might be altered. Most proteins destined for degradation must first undergo covalent conjugation with multiple ubiquitin molecules, which are then recycled following the breakdown of the targeted substrates^{12, 13}. Ubiquitination tags proteins for ATP-dependent hydrolysis by the 26S proteasome, a barrel-shaped multicatalytic proteinase composed of a 20S proteasome functional core^{12, 13} and additional cap-modulator proteins such as PA700, required for the recognition of ubiquitinated proteins^{14, 15}. The nuclear aggregates in polyglutamine repeat diseases may resist degradation, prevent ubiquitin recycling, and/or disrupt the proteasome.

Perturbations in normal proteasome function are associated with increased expression of several highly conserved and structurally-related families of stress-response or heat shock proteins (hsps)¹⁶⁻¹⁹. These proteins function as molecular chaperones—they recognize misfolded proteins and suppress protein aggregation, under both normal and stressed conditions²⁰⁻²². Furthermore, chaperones may maintain proteins in a conformation which allows their appropriate refolding, recognition, and modification by the ubiquitination machinery, or hydrolysis by the proteasome^{21, 22}. The DnaJ (Hsp40) chaperone family promotes cellular protein folding by binding unfolded polypeptides and regulating the activity of members of the DnaK (Hsp70) family^{21, 22}. In Escherichia coli and Saccharomyces cerevisiae, the DnaJ-type and DnaK-type molecular chaperones are also essential for the rapid degradation of normal and misfolded proteins²³⁻²⁶.

HDJ-2/HSDJ has a higher homology to Ydj1 (49% overall identity) than to any other yeast DnaJ homolog^{29, 30}. Based on studies of the yeast Ydjl, the human homolog appears to have four conserved domains that act as functional units^{20, 37}: the DnaJ-domain, a glycine/phenylalanine (G/F) region, a zinc finger-like region, and a conserved carboxy terminus. The J-domain regulates Hsp70 ATPase activity. The function of the G/F domain is unclear, but it is thought to be a spacer between the J-domain and the zinc finger-like region, which is necessary for folding polypeptides once bound. The carboxy terminus binds unfolded polypeptides and is essential for preventing aggregation of the model substrate, rhodanese²⁰.

The ubiquitin-proteasome system is an elaborate mechanism cells have developed to regulate the activities of normal proteins as well as avoid the potentially toxic effects of mutant or misfolded proteins. Given the significance of proper protein turnover, it is not surprising that perturbations in the system have been implicated in the pathogenesis of a number of diseases.

While ubiquitination potentially serves to direct the degradation of ataxin-1, the defect leading to the accumulation of the mutant protein in the affected neurons is, however, less clear. Conjugation of wild-type and mutant ataxin-1 occurs with nearly equal kinetics suggesting the limiting factor in mutant ataxin-1 hydrolysis is not the conjugation of ubiquitin but rather the recognition or hydrolysis of the mutant protein by the proteasome.

The data shown herein demonstrates both the relation between the proteasome and ataxin-1 aggregates and the role of molecular chaperones in SCA1 pathogenesis and provides support for the present invention for the treatment of neurodegenerative disease.

### SUMMARY OF THE INVENTION

An object of the present invention is a method of treating neurodegenerative disease with chaperones or chaperone-like-compounds.

A further object of the present invention is a method for screening for a test compound for chaperone-like activity.

An additional object of the present invention is a method of treating neurodegenerative disease in a mammal by upregulating proteasome activity.

Thus, in accomplishing the foregoing objects there is provided in accordance with one aspect of the present invention a method of treating neurodegenerative disease in a mammal comprising introducing a therapeutic effective amount of a chaperone or chaperone-like-compound into the neurological system of the mammal.

In specific embodiments of the present invention the introducing step includes introducing the chaperone or chaperone-like-compound into the mammal by gene therapy.

In another specific embodiment of the present invention the introducing step includes directly injecting the chaperone or chaperone-like-compound into the mammal.

An alternative embodiment of the present invention includes, a method for screening for a test compound for chaperone-like activity for the treatment of neurodegenerative diseases comprising the steps of introducing the test compound into transfected cells in tissue culture, wherein such transfected cells produce nuclear aggregate inclusions, and measuring the quantity of nuclear aggregate inclusions, wherein a test compound which decreases the quantity of nuclear aggregate inclusions as compared to control cells has chaperone activity.

Another alternative embodiment of the present invention includes, a method for screening for a test compound for chaperone-like activity for the treatment of neurodegenerative diseases comprising the steps of introducing the test compound into an animal which models neurodegenerative disease, allowing said animal to develop, and subsequently measuring the quantity of aggregates in said animal wherein decreased aggregate formation over control animals indicates chaperone-like activity.

A further alternative embodiment of the present invention includes, a method of treating neurodegenerative disease in a mammal comprising the step of introducing a therapeutically effective amount of a compound into said mammal wherein said compound increases the effective concentration of a chaperone in the neurological system.

Another alternative embodiment of the present invention includes, a method of treating neurodegenerative disease in a mammal comprising the step of introducing a therapeutically effective amount of a compound into said mammal wherein said compound increases the effective concentration or enhances the activity of a proteasome in the neurological system.

Other and further objects features and advantages will be apparent from the following description of the presently preferred embodiments of the invention, which are given for the purpose of disclosure when taken in conjunction with the accompanying drawings.

### DESCRIPTION OF THE DRAWINGS

FIGS. 1a, 1b, 1c, 1d and 1e show immunohistochemical localization of 20S proteasome in brainstem neurons from an SCA1 patient and Purkinje cells of transgenic mice. FIGS. 1a and 1b show distribution of the proteasome in neurons of the nucleus pontis centralis from an SCA1 patient and control, respectively. Note the redistribution of the proteasome to the sites of ataxin-1 aggregation. The staining for the proteasome in cerebellar tissue from nontransgenic mice (FIG. 1c) and mice expressing a wild-type ataxin-1 with 30 glutamines (FIG. 1d) is diffuse in the nuclei of Purkinje cells. In contrast, the 20S proteasome colocalizes with ataxin-1 aggregates in mice expressing mutant ataxin-1 with 82 glutamines. FIG. 1e.

FIGS. 2a, 2b, 2c and 2d show immunohistochemical staining of HDJ-2/HSDJ in SCA1 patient neurons and transgenic mice Purkinje cells. These figures show nucleus pontis centralis from an SCA1 patient FIG. 2a and control FIG. 2b; cerebellum from B05 transgenic animal (FIG. 2c) and nontransgenic littermate control (FIG. 2d). HDJ-2/HSDJ is localized mainly to the cytoplasm except for the intranuclear inclusions seen in (FIGS. 2a and 2c).

FIGS. 3a, 3b, 3c and 3d show ubiquitin immunostaining in COS7 cells expressing ataxin-1-GFP and demonstrates the presence of ubiquitin in ataxin-1 aggregates. FIG. 3a shows diffuse staining for ubiquitin in the cytoplasm and the nucleus of nontransfected control cells. The same three cells are shown in FIGS. 3b, 3c, and 3d. In FIG. 3b Ataxin-1-GFP fluorescence is used to identify the ataxin-1 aggregates in the two transfected cells. In FIG. 2c anti-ubiquitin staining (phase contrast) demonstrates that ataxin-1 aggregates are ubiquitin-positive. In FIG. 2d GFP fluorescence is overlaid on phase contrast to demonstrate colocalization of ubiquitin and ataxin-1 aggregates.

FIGS. 4a, 4b and 4c show subcellular localization of 20S proteasome and ataxin-1 in HeLa cells. In FIG. 2a the arrows indicate three cells transfected with ataxin-1, demonstrating ataxin-1 aggregates (red) and the arrow heads point to the three nuclei of non-transfected cells, counter-stained with DAPI. FIG. 4b shows the same cells stained with anti-20S proteasome antibody. Non-transfected cells show diffuse nuclear staining with occasional large foci; transfected cells show proteasome coinciding with ataxin-1 aggregates. FIG. 4c merged signals demonstrating colocalization of ataxin-1 and proteasome.

FIGS. 5a, 5b, 5c, 5e and 5f show colocalization of endogenous HDJ-2/HSDJ and HSP70 with ataxin-1 nuclear aggregates. The distribution of endogenous HDJ-2/HSDJ is shown in FIG. 5a and ataxin-1 (red counter-stained with DAPI) in FIG. 5b. Merger of the two signals as shown in FIG. 5c demonstrates the colocalization of endogenous HDJ-2/HSDJ with the ataxin-1 aggregates. FIGS. 5d, 5e and 5f show Hsp70 in HeLa cells with ataxin-1 aggregates. The distribution of: Hsp70 (green) is seen in FIG. 5d and ataxin-1 (red) is seen in FIG. 5e. Overlay of two signals is seen in FIG. 5f and demonstrates that Hsp70 localizes to ataxin-1 aggregates.

FIGS. 6a, 6b, 6c, 6d and 6e show suppression of ataxin-1 aggregation in cells overexpressing HDJ2/HSDJ. In FIG. 6a the bars represent the percentage of cells with nuclear aggregates after cotransfection with ataxin-1 and control vector, or ataxin-1 and either of three HDJ-2/HSDJ constructs: wild-type (HDJ-2/HSDJ) and two deletion mutants (Δaa9-107, or Δaa9-46). The data were generated from two independent experiments and the total number of cotransfected cells used to calculate the frequency of aggregates is: 695 for ataxin-1 and vector control, 1302 for ataxin-1 and HDJ-2/HSDJ, 841 for ataxin-1 and Δaa9-107, and 550 for ataxin-1 and Δaa9-46 (means and s.e.m. are shown). A significant decrease in aggregate frequency is noted in cells transfected with wild-type chaperone compared to vector and either of the two deletion mutants (ANOVA F=24.5, DF=3.8 and p=0.0002). No significant change in frequency of aggregation is noted upon transfection with either deletion mutants. FIG. 6b shows the distribution of the staining pattern of ataxin-1 after transfection. FIG. 6b indicates that, in the presence of wild-type HDJ-2/HSDJ, more cells have diffuse/micropunctate nuclear staining pattern, than large nuclear aggregates (ANOVA F=36.4, DF=6.24 and p<0.001). The frequency of cells with each staining pattern is plotted for each cotransfection. FIGS. 6c, 6d and 6e show examples of the various staining patterns in the presence of HDJ-2/HSDJ.

FIGS. 7A through 7D show proteasome inhibition in transfected HeLa cells.

FIGS. 8A through 8D show the effect of β-lactone on steady-state levels of ataxin-1. FIG. 8A whole lysate; FIG. 8B detergent soluble fraction; FIG. 8C detergent insoluble fraction; FIG. 8D detergent insoluble fraction denaturing 6xHis pull-down.

FIGS. 9A through 9F showing that Purkinje cells in double mutant animals contain ubiquitinated material.

FIGS. 10A through 10I show Purkinje cell vacuolation and cell body displacement in the cerebellum.

The drawings are not necessarily to scale. Certain features of the invention may be exaggerated in scale or shown in schematic form in the interest of clarity and conciseness.

### DETAILED DESCRIPTION OF THE INVENTION

It is readily apparent to one skilled in the art that various substitutions and modifications may be made to the invention disclosed herein without departing from the scope and spirit of the invention.

As used in here, the term “chaperone” refers to those proteins which are produced in eucaryotic cells that either help other proteins to fold or allow misfolded proteins to refold into proper structure. A variety of such proteins are known in the art. For example, Hsp60, Hsp40, and Hsp70 are examples of such proteins. The skilled artisan knows how to determine such proteins.

The term “chaperone-like-compound” is used in the present invention to refer to those proteins or chemical compounds which show chaperone-like activity. More specifically it refers to those compounds which show the ability to prevent aggregation of proteins in the cells of the nervous system.

As used herein, the term “gene therapy” has the meaning commonly known in the art. This includes any method known in gene therapy where a gene has been inserted into an organism. In many cases, using gene therapy and appropriate delivery vehicles, the gene can be targeted to specific tissues.

As used herein, the term “neurodegenerative disorders” refers to those neurodegenerative disorders which have the characteristic of insoluble aggregates in the cells of the nervous system. Some examples of these type of diseases include Alzheimer disease, Parkinson disease, the prion disorders, Huntington disease (HD), dentatorubral-pallidoluysian atrophy (DRPLA), spinocerebellar ataxia type 1 and 3 (SCA1 and SCA3) and any other neurodegenerative diseases caused by CAG repeat expansion.

As used herein, the term “protein aggregate” includes protein misfolding and the clumping of proteins. In both cases, the protein is not degraded normally.

As used herein, the term “transfected cells” refers to those cells in which a foreign gene has been inserted into the cells, and is expressed in said cells.

One aspect of the present invention is a method of treating neurodegenerative disease in a mammal comprising introducing a therapeutic effective amount of a chaperone or chaperone-like-compound into the neurological system of the mammal.

In specific embodiments of the present invention the introducing step includes injecting the chaperone or chaperone-like-compound into the mammal by gene therapy.

In a further specific embodiment of the present invention the introducing step includes introducing the chaperone or chaperone-like-compound into the mammal.

An alternative embodiment of the present invention includes, a method for screening for a test compound for chaperone-like activity for the treatment of neurodegenerative diseases comprising the steps of introducing the test compound into transfected cells in tissue culture, wherein such transfected cells produce nuclear aggregate inclusions, and measuring the quantity of nuclear aggregate inclusions, wherein a test compound which decreases the quantity of nuclear aggregate inclusions as compared to control cells has chaperone activity.

Another alternative embodiment of the present invention includes, a method for screening for a test compound for chaperone-like activity for the treatment of neurodegenerative diseases comprising the steps of introducing the test compound into an animal which models neurodegenerative disease, allowing said animal to develop, and subsequently measuring the quantity of aggregates in said animal wherein decreased aggregate formation over control animals indicates chaperone-like activity.

A further alternative embodiment of the present invention includes, a method of treating neurodegenerative disease in a mammal comprising the step of introducing a therapeutically effective amount of a compound into said mammal wherein said compound increases the effective concentration of a chaperone in the neurological system.

Another alternative embodiment of the present invention includes, a method of treating neurodegenerative disease in a mammal comprising the step of introducing a therapeutically effective amount of a compound into said mammal wherein said compound increases the effective concentration of a proteasome in the neurological system.

Another alternative is to enhance the activity of the proteasome such that it is more efficient at degrading misfolded proteins.

It is important to recognize that the compounds (chaperones, chaperone-like-compounds and compounds that increase the effective concentration of proteasome or enhance its activity) will be used in a pharmaceutically acceptable mode of delivery to the source of the tissue. This can include in vitro, in vivo or ex vivo administration.

### Therapeutic Effective Amount

As used in the present invention, a compound will be considered therapeutically effective if it decreases, delays or eliminates the onset of the neurological disease or decreases, delays or eliminates protein misfolding, delays or eliminates the formation of insoluble aggregates in the neurological system. A skilled artisan readily recognizes that in many of these cases the compound may not provide a cure but may only provide partial benefit. A physiological change having some benefit is considered therapeutically beneficial. Thus, an amount of compound which provides a physiological change is considered an “effective amount” or a “therapeutic effective amount”.

A compound, molecule or composition is said to be “pharmacologically acceptable” if its administration can be tolerated by a recipient mammal. Such an agent is said to be administered in a “therapeutically effective amount” if the amount administered is physiologically significant. An agent is physiologically significant if its presence results in technical change in the physiology of a recipient mammal. For example, in the treatment of neurological disorders of the present invention, a compound would be therapeutically effective if it (i) inhibits protein misfolding and/or the formation of or decreases the amount of the insoluble aggregation in the nervous system or (ii) delays the onset of the symptoms of the neurological disorder.

### Dosage and Formulation

The chaperones, chaperone-like-compounds and compounds that increase the effective concentration of proteasome (active ingredients) of this invention can be formulated and administered to inhibit a variety of disease and nondisease states by any means that produces contact of the active ingredient with the agent or its site of action in the body of a mammal. The compounds can be administered by any conventional means available for use in conjunction with pharmaceuticals, either as individual therapeutic active ingredients or in a combination of therapeutic active ingredients. They can be administered alone, but are generally administered with a pharmaceutical carrier selected on the basis of the chosen route of administration and standard pharmaceutical practice.

Dosages for other uses will vary depending on the physical effect desired. These relationships are generally known in the art for compounds having similar effects and can be readily determined by the skilled artisan.

The dosage administered will be a therapeutically effective amount of active ingredient and will, of course, vary depending upon known factors such as the pharmacodynamic characteristics of the particular active ingredient and its mode and route of administration; age, sex, health and weight of the recipient; nature and extent of symptoms; kind of concurrent treatment, frequency of treatment and the effect desired. A daily dosage (therapeutic effective amount) of active ingredient can be given in divided doses 2 to 4 times a day or in sustained release form.

Dosage forms (compositions) suitable for internal administration contain from about 1.0 to about 500 milligrams of active ingredient per unit. In these pharmaceutical compositions, the active ingredient will ordinarily be present in an amount of about 0.05-95% by weight based on the total weight of the composition.

The active ingredient can be administered orally in solid dosage forms such as capsules, tablets and powders, or in liquid dosage forms such as elixirs, syrups, emulsions and suspensions. The active ingredient can also be formulated for administration parenterally by injection, rapid infusion, nasopharyngeal absorption or dermoabsorption. The agent may be administered intramuscularly, intravenously, or as a suppository. Additionally, gene therapy modes of introduction can be used to target the introduction of the compound. The skilled artisan readily recognizes that the dosage for this method must be adjusted depending on the efficiency of delivery.

Gelatin capsules contain the active ingredient and powdered carriers such as lactose, sucrose, mannitol, starch, cellulose derivatives, magnesium stearate, stearic acid, and the like. Similar diluents can be used to make compressed tablets. Both tablets and capsules can be manufactured as sustained release products to provide for continuous release of medication over a period of hours. Compressed tablets can be sugar coated or film coated to mask any unpleasant taste and protect the tablet from the atmosphere, or enteric coated for selective disintegration in the gastrointestinal tract.

Liquid dosage forms for oral administration can contain coloring and flavoring to increase patient acceptance.

In general, water, a suitable oil, saline, aqueous dextrose (glucose), and related sugar solutions and glycols such as propylene glycol or polyethylene glycols are suitable carriers for parenteral solutions. Solutions for parenteral administration contain preferably a water soluble salt of the active ingredient, suitable stabilizing agents and, if necessary, buffer substances. Antioxidizing agents such as sodium bisulfate, sodium sulfite or ascorbic acid either alone or combined are suitable stabilizing agents. Also used are citric acid and its salts and sodium EDTA. In addition, parenteral solutions can contain preservatives such as benzalkonium chloride, methyl- or propyl-paraben and chlorobutanol. Suitable pharmaceutical carriers are described in Remington's Pharmaceutical Sciences, a standard reference text in this field.

Additionally, standard pharmaceutical methods can be employed to control the duration of action. These are well known in the art and include control release preparations and can include appropriate macromolecules, for example polymers, polyesters, polyaminoacids, polyvinyl, pyrolidone, ethylenevinylacetate, methyl cellulose, carboxymethyl cellulose or protamine sulfate. The concentration of macromolecules as well as the methods of incorporation can be adjusted in order to control release. Additionally, the agent can be incorporated into particles of polymeric materials such as polyesters, polyaminoacids, hydrogels, poly (lactic acid) or ethylenevinylacetate copolymers. In addition to being incorporated, these agents can also be used to trap the compound in microcapsules.

Useful pharmaceutical dosage forms for administration of the compounds of this invention can be illustrated as follows.

Capsules: Capsules are prepared by filling standard two-piece hard gelatin capsulates each with 100 milligram of powdered active ingredient, 175 milligrams of lactose, 24 milligrams of talc and 6 milligrams magnesium stearate.

Soft Gelatin Capsules: A mixture of active ingredient in soybean oil is prepared and injected by means of a positive displacement pump into gelatin to form soft gelatin capsules containing 100 milligrams of the active ingredient. The capsules are then washed and dried.

Tablets: Tablets are prepared by conventional procedures so that the dosage unit is 100 milligrams of active ingredient. 0.2 milligrams of colloidal silicon dioxide, 5 milligrams of magnesium stearate, 275 milligrams of microcrystalline cellulose, 11 milligrams of cornstarch and 98.8 milligrams of lactose. Appropriate coatings may be applied to increase palatability or to delay absorption.

Injectable: A parenteral composition suitable for administration by injection is prepared by stirring 1.5% by weight of active ingredients in 10% by volume propylene glycol and water. The solution is made isotonic with sodium chloride and sterilized.

Suspension: An aqueous suspension is prepared for oral administration so that each 5 millimeters contain 100 milligrams of finely divided active ingredient, 200 milligrams of sodium carboxymethyl cellulose, 5 milligrams of sodium benzoate, 1.0 grams of sorbitol solution U.S.P. and 0.025 millimeters of vanillin.

### EXAMPLE 1

### Plasmids

A human SCA1 cDNA containing 82 CAG repeats was subcloned in pcDNA3.1/HIS-C (Invitrogen)⁴². The same cDNA was subcloned inframe into PEGFP (Clonetech) to generate an ataxin-1/GFP fusion construct. After the cDNA was subcloned the polyglutamine repeat size was confirmed by DNA sequence analysis. The CAG repeat in the pcDNA 3.1 vector expanded to 92 while the CAG repeat in the GFP construct remained unchanged.

Full-length human HDJ-2/HSDJ and HDJ-2/HSDJ mutants Δ250 (deletion of aa 9-46) and Δ450 (deletion of aa9-107)³² were subcloned in frame into the pFLAG-CMV-2vector (Kodak). The primers HDJ2-FOR (5′-aataagaatgcggccgcgatggtgaaagaaacaacttac-3′) and HDJ2-REV (5′-gaatttgctgaaccattccaggtc-3′) were used to PCR amplify the 5′ end of HDJ-2/HSDJ containing an inframe Not I site. The PCR product was cut with Notl and EcoRl and subcloned into pFLAG-CMV-2. This construct was digested with EcoRl and Xbal to insert the 3′ HDJ-2/HSDJ EcoRl/Xbal sequence. The constructs were confirmed by DNA sequence analysis.

### EXAMPLE 2

### Immunohistochemistry and Immunofluorescence

Immunohistochemical staining was performed using monoclonal or polyclonal antibody on human and mouse brain sections by methods known in the art⁷. The following antisera used to stain brain tissue were purchased from StressGen: rabbit polyclonal anti-Hsp25 (SPA-801), mouse monoclonal anti-Hsp27 (SPA-800), mouse monoclonal anti-Hsp60 (SPA-806), rabbit polyclonal anti-Hsp90α (SPA-771), mouse monoclonal anti-Hsp70 (SPA-810), mouse monoclonal anti-Hsp70/Hsc70 (SPA-882), and rabbit polyclonal anti-Hsp110 (SPA-1103). Mouse and human HDJ-2/HSDJ were detected with mouse monoclonal anti-HDJ-2/DNAJ Ab-1-clone KA2A5.6 (Neomarkers). The 20S proteasome, PA700 and P31 were visualized with rabbit polyclonal anti-20S proteasome⁴³, chicken polyclonal anti-PA700, and rabbit polyclonal anti-P31.

Transient expression of ataxin-1 and HDJ-2/HSDJ in COS7 and HeLa cells was accomplished by transfection with Lipofectamine™ Reagent (Life technologies, Inc.) in 35 mm tissue culture plates containing sterile coverslips. Forty-eight hours after transfection, cells were fixed at 4° C. for 30 min in 4% formaldehyde in PEM (80 mM K-PIPES, pH 6.8, 5 mM EGTA pH 7.0, 2 mM MgCl₂), quenched in 1 mg/ml NaBH₄ in PEM, and permeabilized for 30 min in 0.5% Triton X-100 in PEM. The coverslips were blocked for 1 hour at room temperature (RT) in 5% non-fat dry milk (Bio-Rad) in 50 mM Tris-HCl pH 7.5, 150 mM NaCl, 0.05% Tween20 (TBS-T), then incubated for 60 min at RT with rabbit polyclonal antibodies (1:1000) recognizing ataxin-1 (11750VII)⁴⁴. Mouse monoclonal antibodies (1:1000) M2 (Kodak) recognizing the FLAG epitope in the HDJ-2/HSDJ constructs were used to stain recombinant HDJ-2/HSDJ. Subsequently, cells were incubated with either anti-rabbit-Texas Red or anti-mouse-FITC (Vector Laboratories) (1:600), counterstained for 1 min in TBS-T containing DAPI (1 μg/ml) then mounted in antifade solution (Vectashield mounting media, Vector Laboratories). Hsp70 was detected with mouse monoclonal anti-Hsp70 (1:500) (StressGen). Endogenous HDJ-2/HSDJ was detected with mouse monoclonal anti-HDJ-2/DNAJ (1:200) (Neomarkers). The 20S proteasome was visualized with rabbit polyclonal anti-20S proteasome⁴³ (1:500) and ataxin-1 colocalization was detected using mouse monoclonal anti-Xpress (1:1000) recognizing the Xpress epitope in pcDNA 3.1 (Invitrogen). Ubiquitin was visualized with mouse monoclonal anti-ubiquitin (1:200) (Novo Castra) following avidin-biotin peroxidase complex (ABC) reaction according to manufacturer's protocol (Vector Laboratories) and co-localized with ataxin-1-GFP by immunofluorescence.

### EXAMPLE 3

### Quantitative Analysis of Ataxin-1 Aggregate Formation in HeLa Cells

Duplicate slides were graded blindly in two independent trials. Each slide had over 200 cells cotransfected with HDJ2/HSDJ and ataxin-1; cells were categorized by their staining pattern of ataxin-1 as either 1) diffuse, 2) micropunctate, or 3) large aggregates. The total number of cotransfected cells graded for aggregates were: 1302 for ataxin-1 and HDJ2/HSDJ; 550 for ataxin-1 and Δ250; 841 for ataxin-1 and Δ450, and 695 for ataxin-1 and empty vector. Frequency of aggregate formation was computed for two independent experiments and expressed as the mean±s.e.m. Statistical analyses (ANOVA) were performed using SPSS software version 6.1.

### EXAMPLE 4

### The Proteasome Colocalizes with Ataxin-1 Aggregates

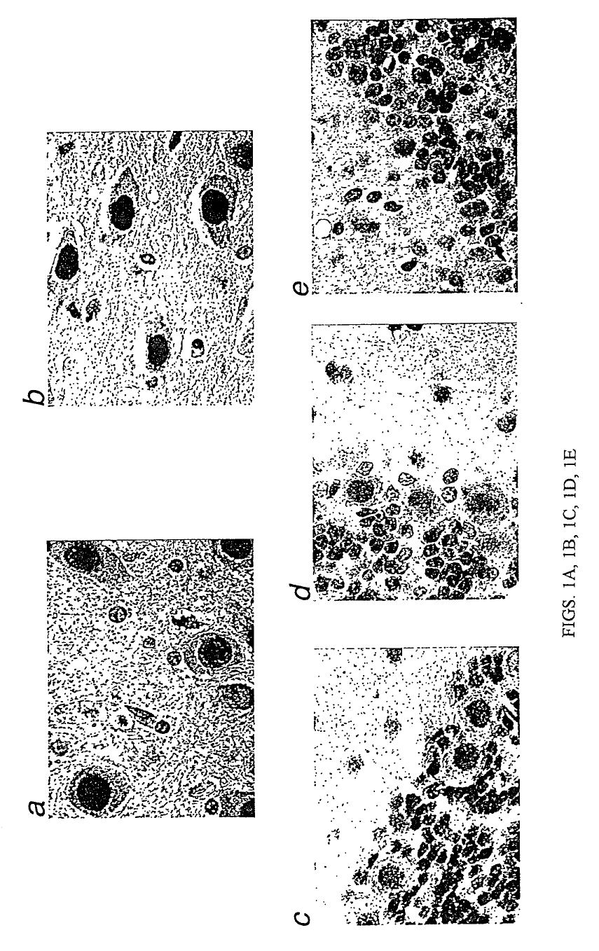

To ascertain proteasome distribution in nuclei containing the ubiquitin-positive ataxin-1 inclusions, brain tissue from an SCA1-affected region, the nucleus pontis centralis, by immunohistochemistry were analyzed. Ataxin-1 nuclear aggregates are intensely immunoreactive to anti-20S proteasome polyclonal antisera, and show a dense accumulation of punctate structures throughout the approximately 2 μm inclusion (FIG. 1a). The remainder of the nucleus shows diffuse staining, but less than that observed in neuronal nuclei from an unaffected control (FIG. 1b).

Also examined were Purkinje cells of transgenic mice expressing either the wild-type SCA1 allele (A02 line containing 30 glutamines [30Q]) or the mutant allele (B05 line containing 82Q)²⁷. In the Purkinje cells of nontransgenic control mice and mice from the A02 line, the distribution of the 20S proteasome was diffusely nuclear with faint cytoplasmic staining (FIGS. 1c, d) In B05 Purkinje cells, however, the 20S proteasome is localized to a single large nuclear inclusion (FIG. 1e). As observed in the neurons of the SCA1 patient, the 20S proteasome staining was concentrated in or around the nuclear inclusion. Staining in the remainder of the nucleus was fainter than that seen in the nontransgenic control or the A02 line. Thus, in both an SCA1 patient and a transgenic mouse model of ataxia, the 20S proteasome complex is redistributed in the nuclei of affected neurons to the sites of ataxin-1 protein aggregation. The distribution of the PA700 regulatory subunit and the P31 cap modulator of the 26S proteasome^{14, 28} were also altered to colocalize with ataxin-1 aggregates in the SCA1 patient and transgenic mice.

### EXAMPLE 5

### Ataxin-1 Nuclear Aggregates are Positive for HDJ-2/HSDJ

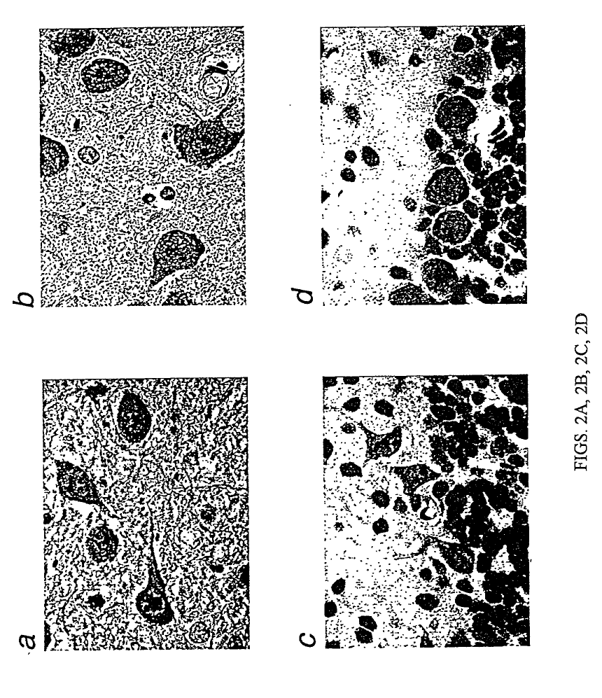

Given the role of eukaryotic DnaJ homologs in protein folding, ubiquitin-dependent protein degradation, and aggregation suppression^{21, 22}. The expression and subcellular localization of a human DnaJ homolog, HDJ-2/HSDJ, in brain tissue from an SCA1 patient and transgenic mice were examined. HDJ-2/HSDJ is one of three mammalian DnaJ homologs cloned to date^{29, 30} and most closely resembles the yeast Ydj-1 protein³¹. Upon immunostaining, it was found that the ataxin-1 nuclear inclusions in the nucleus pontis centralis were HDJ-2/HSDJ-positive (FIG. 2a). HDJ2/HSDJ localized mainly to the cytoplasm except for the nuclear inclusion. In Purkinje cells of transgenic mice expressing mutant ataxin-1, the mouse HDJ-2 homolog was similarly cytoplasmic except for colocalization with the nuclear inclusion (FIG. 2c). Purkinje cells in the nontransgenic controls showed predominantly cytoplasmic staining (FIG. 2d).

Since members of the Hsp40 family of molecular chaperones such as HDJ-2/HSDJ often function in concert with Hsp70 chaperones^{19, 21, 22}, the expression and subcellular distribution of inducible Hsp70 was examined. Hsp70 immunostaining was undetectable in the nucleus pontis centralis of the SCA1 patient and control. Similarly, Hsp70 was undetectable in Purkinje cells of A02, B05, and nontransgenic control mice. These results indicated that ataxin-1 nuclear inclusions do not elicit the stress response necessary to increase the expression of inducible Hsp70.

Also examined was the expression and subcellular distribution of the constitutive member of the Hsp70 family, Hsc70, using an antibody that recognizes both Hsp70 and Hsc70. In the nucleus pontis centralis from an SCA1 patient, Hsc70 was detected in the ataxin-1 nuclear aggregates and there was faint staining of the nuclear inclusion in Purkinje cells of B05 mice. The staining pattern of Hsc70 was considerably weaker than that of HDJ-2/HSDJ in both of these tissues. The staining pattern of additional hsps—including Hsp25/27, Hsp60, the neuronal form of Hsp90 (Hsp90α), and Hsp110—indicated that none of these proteins colocalize with the ataxin-1 aggregates.

### EXAMPLE 6

### The Proteasome and Ataxin-1 Aggregates in Transfected Cells

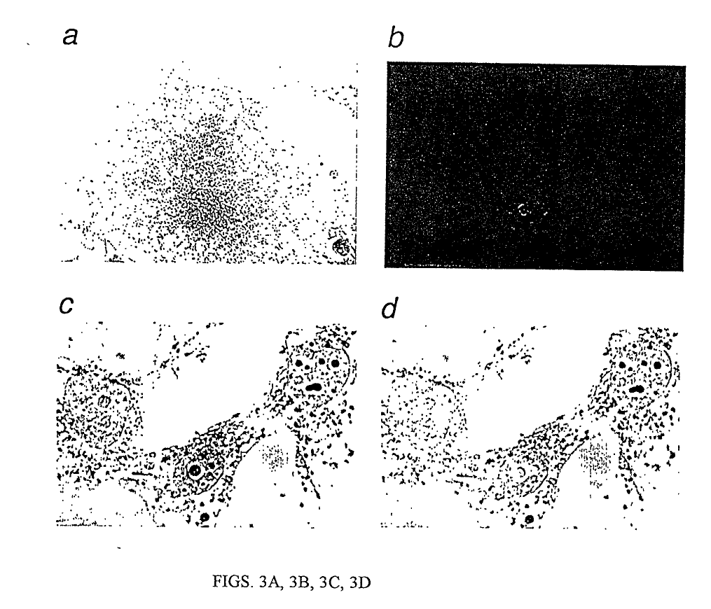

Because HDJ-2/HSDJ and the 20S proteasome were redistributed to ubiquitin-positive inclusions in the affected cells of transgenic mice and the SCA1 patient, these proteins were examined in transfected cells. The cultured cells are amenable to manipulation and provide a model of phenotypic abnormalities observed in vivo. Cells were transfected with a construct containing a fusion of ataxin-1 and green fluorescent protein (GFP) and then stained for ubiquitin. Nontransfected cells display diffuse ubiquitin staining (FIG. 3a), but the ataxin-1 transfected cells display ubiquitin-positive aggregates (FIG. 3c). Colocalization of ubiquitin and GFP-ataxin-1 is demonstrated by overlapping the bright field image with that generated by immunofluorescence (FIGS. 3b, d).

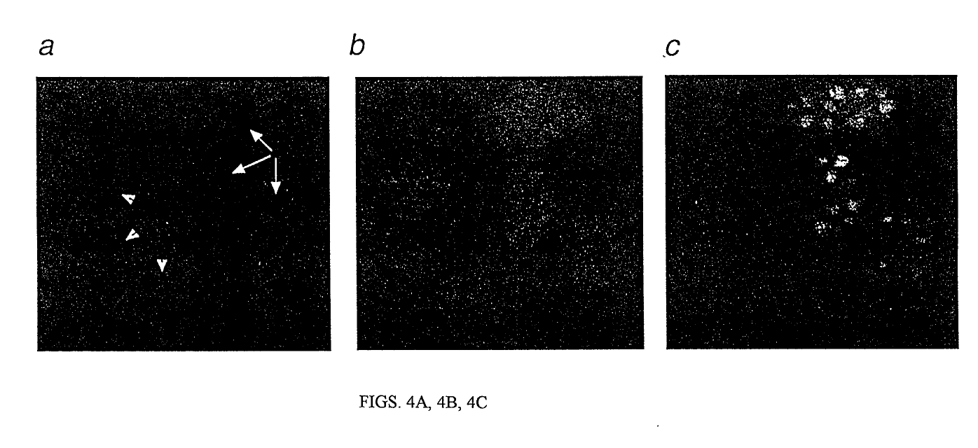

To determine if the ataxin-1 aggregates were also positive for the 20S proteasome, HeLa cells transfected with ataxin-1 were costained for ataxin-1 and the endogenous 20S proteasome. As shown in FIG. 4, the 20S proteasome staining pattern in nontransfected cells is primarily punctate in the nucleus with a small number of large, irregularly-shaped foci (FIG. 4). Transfecting the cells with ataxin-1 alters the staining pattern of the 20S proteasome such that it clearly coincides with the nuclear aggregates formed by ataxin-1 (FIG. 4c). Taken together, these data indicate that a protein (or proteins) within the aggregates is ubiquitinated and targeted for hydrolysis by the proteasome. The abnormal nuclear distribution of the 20S proteasome suggests that although the proteasome localizes to the ataxin-1 aggregates, it is not able to degrade proteins within them.

### EXAMPLE 7

### Chaperones in Ataxin-1 Aggregates in Transfected Cells

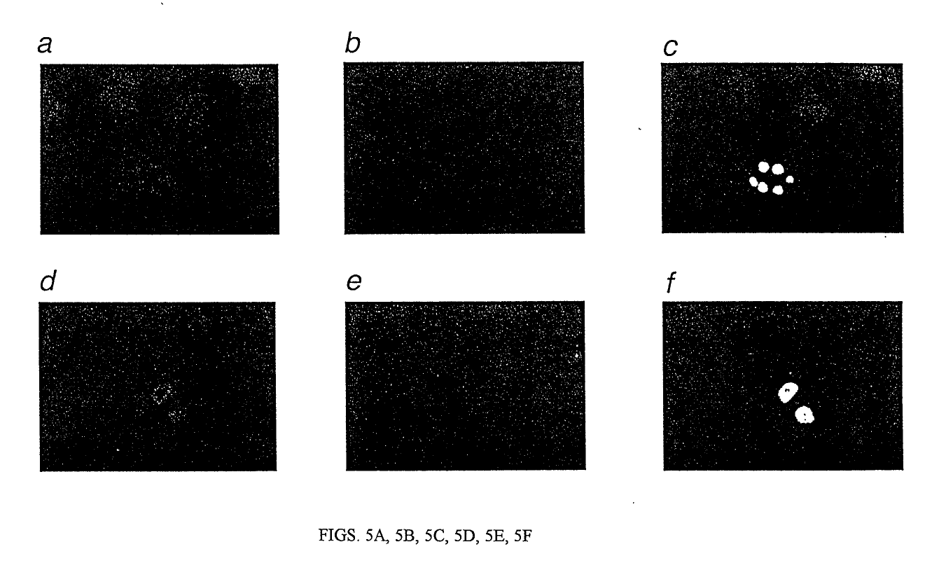

The subcellular distribution of HDJ-2/HSDJ in HeLa cells transfected with ataxin-1 was examined. Endogenous HDJ-2/HSDJ in nontransfected HeLa cells was predominantly cytoplasmic with limited nuclear staining. Cells transfected with ataxin-1 showed stronger nuclear staining, with clear colocalization of HDJ-2/HSDJ to the aggregates (FIGS. 5a, b, c). Thus, the non-neuronal cell line reproduces the targeting of HDJ-2/HSDJ to ataxin-1 aggregates observed in vivo.

As expected, endogenous Hsp70 was not detected by indirect immunofluorescence in nontransfected HeLa cells. Conversely, Hsp70 staining was evident in cells transfected with ataxin-1, but only in that subset containing large nuclear inclusions. In the latter case, colocalization of Hsp70 and the ataxin-1 nuclear aggregates were seen. (FIGS. 5d, e, f). These data show that Hsp70 is upregulated in cells forming large nuclear aggregates.

### EXAMPLE 8

### Chaperone Overexpression Reduces Ataxin-1 Aggregation

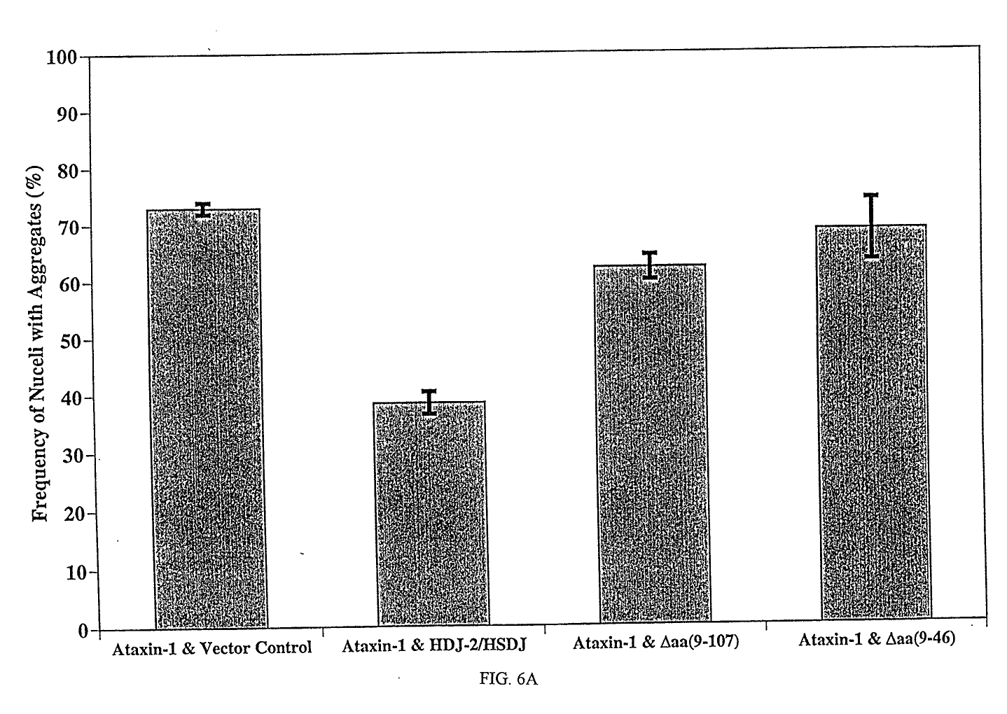

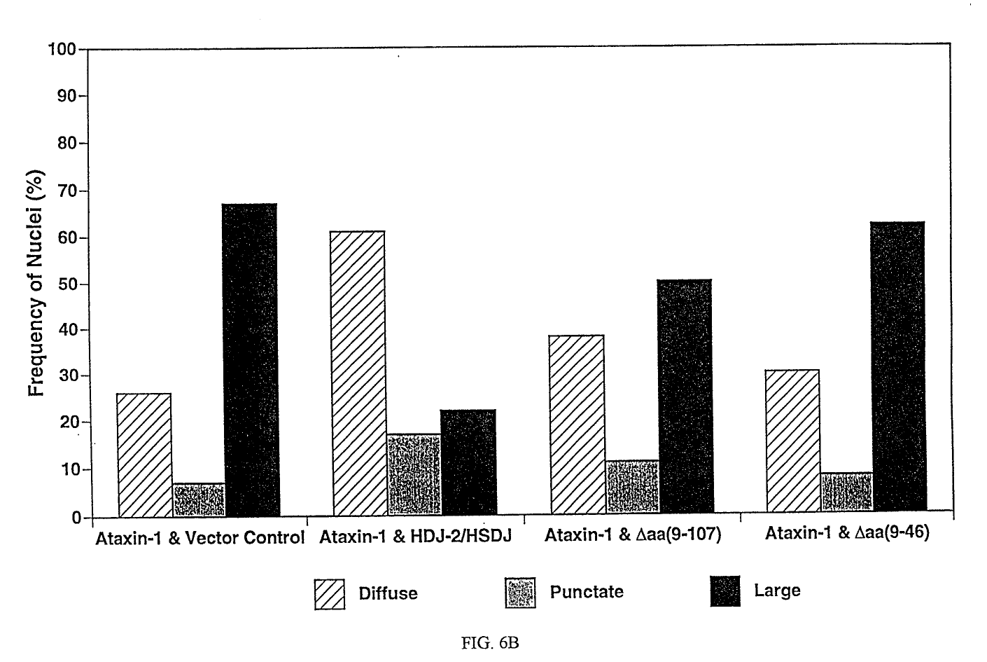

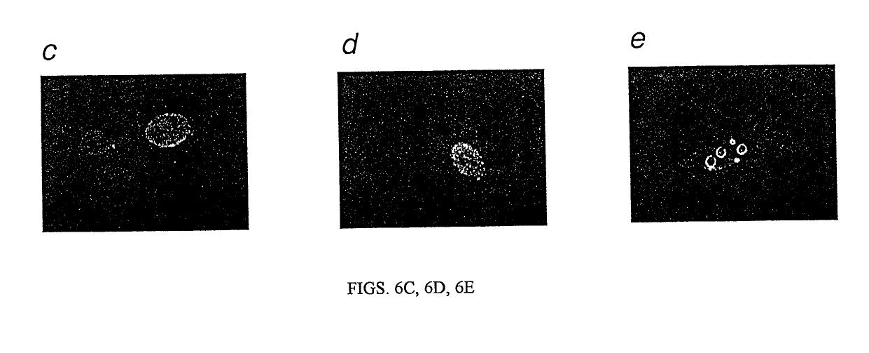

The ability of HDJ-2/HSDJ to function as a molecular chaperone and moderate ataxin-1 aggregation in HeLa cells was tested. The suppression of protein aggregation by a eukaryotic DnaJ protein in vitro requires a relatively large (approximately 10-fold) molar excess of DnaJ protein²⁰. The endogenous DnaJ protein in cells containing ataxin-1 aggregates may not be present at sufficient levels to succeed in suppressing aggregate formation. Tang et al. demonstrated that overexpression of HDJ-2/HSDJ effectively suppressed the formation of nuclear aggregates containing a mutant steroid receptor³². Although ataxin-1 is not a steroid receptor, the Tang reference is a suggestion to try a similar approach. HDJ-2/HSDJ was overexpressed by transfection in HeLa cells and the cells analyzed for the staining pattern of ataxin-1. When HeLa cells were cotransfected with ataxin-1 [92Q] and HDJ-2/HSDJ, ataxin-1 aggregation decreased: while approximately 70% of the cells transfected with ataxin-1 and plasmid vector had nuclear aggregates, less than 40% of cells expressing ataxin-1 and HDJ-2/HSDJ were aggregate-positive (FIG. 6a). No significant decrease in ataxin-1 aggregation in cells coexpressing ataxin-1 and either of two J-domain mutants of HDJ-2/HSDJ (Aaa9-46 or Aaa9-107) was observed. Analysis of variance (ANOVA) revealed differences in the frequency of ataxin-1 aggregation (F=24.5, DF=3.8 and p=0.0002) among the four groups analyzed. The co-expression of wild-type HDJ-2/HSDJ in ataxin-1 transfected cells was responsible for this variation. None of the other group pairs were significantly divergent. Moreover, the distribution of cells containing micropunctate versus large aggregates differed between the HDJ-2/HSDJ-expressing cells and cells expressing vector control or either deletion mutation (FIG. 6b). ANOVA demonstrated a significant correlation between size category of aggregates and expression of HDJ-2/HSDJ (F=36.4, DF=6, 24, and p<0.001). Together, these results indicate that overexpression of a particular molecular chaperone can suppress the aggregation of mutant ataxin-1 in vivo. When sufficient amounts of HDJ-2/HSDJ are targeted to the nucleus in response to ataxin-1 expression, ataxin-1 aggregation is subdued. This protective effect is dependent on the presence of the DnaJ-domain within HDJ-2/HSDJ.

### EXAMPLE 9

### Experiments in Transgenic Mice

The SCA1 transgenic mice provide an excellent animal model for the human disease. In Purkinje neurons of these mice, ataxin-1 aggregates localize with chaperones, and appear to sequester the proteasome. Because of the finding that overexpression of the HDJ-2/HSDJ chaperone decreases ataxin-1 aggregation in cell culture, new transgenic mice that overexpress HDJ-2/HSDJ in Purkinje cells were generated. These mice were generated by expressing the HDJ-2/HSDJ gene under the control of a promoter that directs expression selectively to Purkinje cells. After birth, these transgenic mice are mated with the SCA1 transgenic mice. In this manner the Purkinje cells that express the mutant ataxin-1 have high levels of the HDJ-2/HSDJ chaperone. The clinical course and pathology of these doubly transgenic mice are characterized to document the positive effect of chaperone overexpression in vivo.

### EXAMPLE 10

### Experiments in Cell Culture

The purpose of these experiments is to use cells in culture (cell lines) to screen for a large number of compounds that will modulate the activity of chaperones and the proteasome. The ease of using a cell culture-based assay allows the rapid screening of hundreds of compounds simultaneously. Compounds that prove to modulate the chaperone/proteasome activity such that ataxin-1 misfolding and/or aggregation are reduced or eliminated are then used to develop and test medications that can be used in vivo.

New cell lines were developed to control the levels of mutant ataxin-1 using the tetracycline-regulatable gene expression system (Tet-On™, Clontech). With this system the normal state of the cell is maintained until induction by adding tetracycline. When induced, mutant ataxin-1 is expressed at high levels. Because mutant ataxin-1 is prone to misfolding, it gradually forms aggregates that are observed as they develop. The time of induction is precisely monitored. The modulation of mutant protein aggregation in these cell lines in the presence of an array of compounds known to effect proteasome and/or chaperone function are monitored. These compounds are added to the cells either before or after induction of ataxin-1 expression to determine when is the ideal time to intervene and prevent aggregation.

### EXAMPLE 11

### Proteasome Inhibition Leads to Increased Aggregation and Accumulation of Detergent Insoluble Ataxin-1

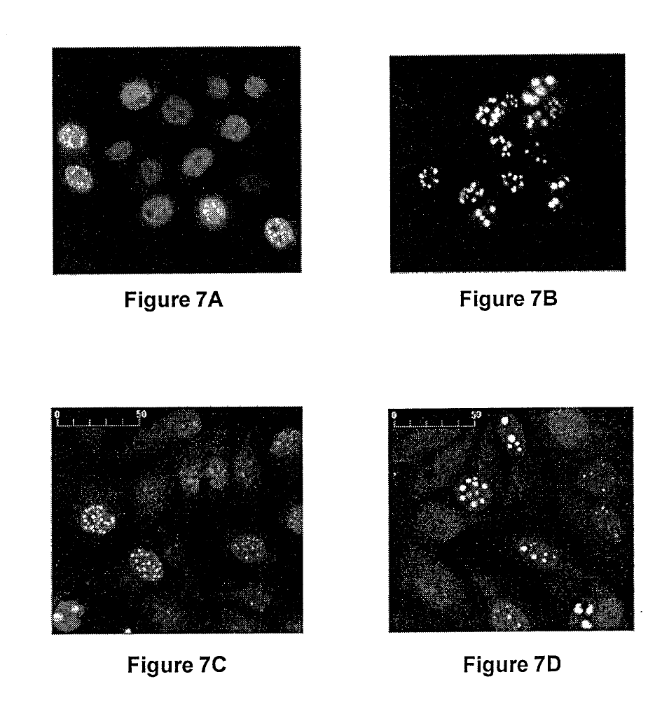

Full length mutant ataxin-1 [82Q] readily aggregates in subnuclear structures when overexpressed in tissue culture cells and these aggregates alter the staining pattern of the 20S proteasome. The abnormal distribution of the proteasome indicates that it is targeting the inclusions in a attempt to degrade the aggregated protein. The effect of proteasome inhibitors on the aggregation of GFP-ataxin-1 [82Q] in transfected HeLa cells was examined. The protease inhibitor clasto-Lactacystin β-lactone specifically prevents protein breakdown by the proteasome, without inhibiting lysosomal degradation. Proteasome inhibition by β-lactone had a dramatic effect on ataxin-1 aggregation. In contrast to the 71% of mock treated cells which had nuclear aggregates, 97% of the proteasome inhibitor treated cells were aggregate positive (FIGS. 7A through 7D). Moreover, the distribution of cells containing the large aggregates was also dramatically increased compared to controls. Only a small percentage of treated cells had a diffuse staining pattern or contained micropunctate structures. Therefore, proteasome inhibitor treatment led to an increase in both frequency and size of nuclear aggregates. This enhancement is most likely due to the increased nuclear concentration of misfolded proteins which are not being properly degraded. A similar effect was seen with a second, less potent, proteasome inhibitor MG132 (CBZ-LLLAL).

### EXAMPLE 12

### Effect of Proteasome Inhibitors on Ataxin-1 Degradation

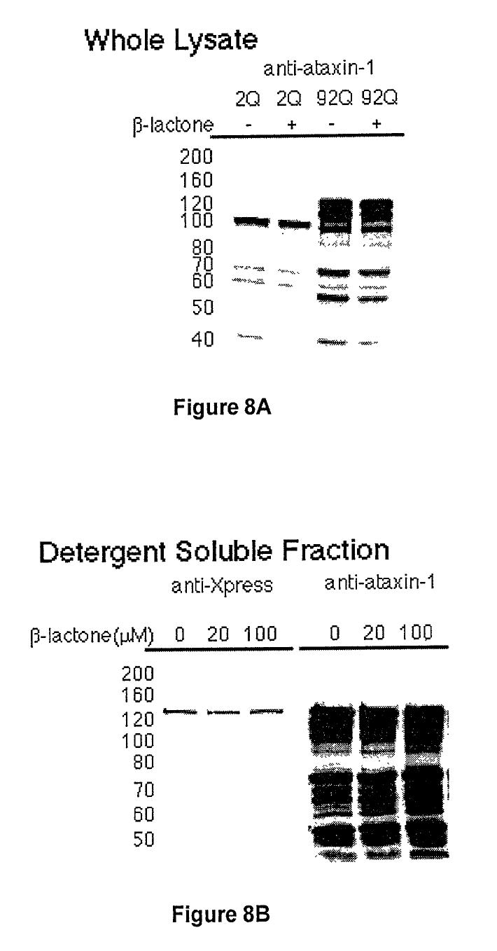

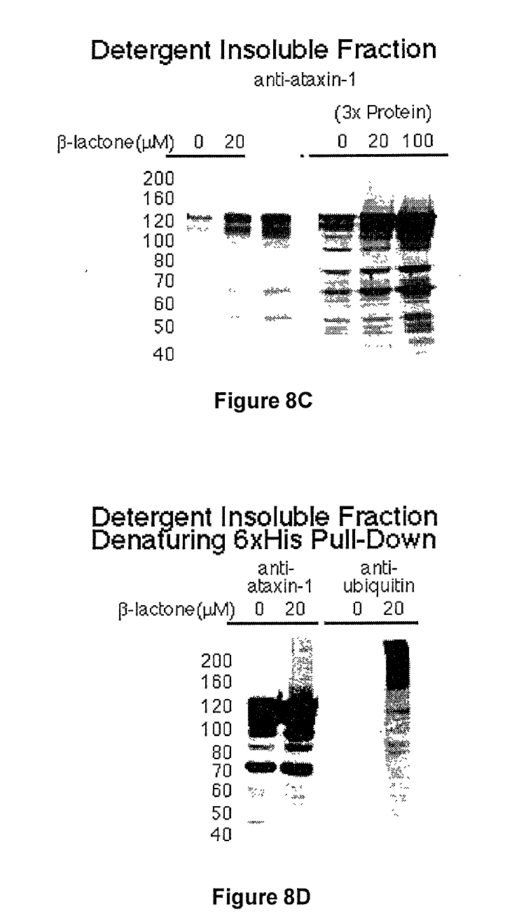

The effect of β-lactone on the steady-state levels of ataxin-1 was assessed by immunoblot analysis (FIGS. 8A through 8D). HeLa cells expressing mutant ataxin-1 [92Q] were treated with either different concentrations of β-lactone or DMSO (dimethyl sulfoxide) control. Cells lysates were separated into detergent soluble and insoluble fractions and then immunoblotted with ataxin-1 antibody. With cell equivalents loaded per lane, it is clear that β-lactone treatment leads to a marked accumulation of the detergent-insoluble form of ataxin-1 suggesting its degradation is via the proteasome pathway. Increasing the protein concentration per lane and extending the exposure time, a high molecular weight smear punctated by discrete bands is evident. Interestingly, the steady state levels of the detergent soluble form of ataxin-1 appeared unchanged in the presence of β-lactone. Additionally, the higher molecular weight smear was never seen in the detergent soluble fraction.

### EXAMPLE 13

### Ataxin-1 is Polyubiquitinated

Upon close examination, the ataxin-1 immunoreactive smear contains a ladder of bands regularly spaced at intervals of Å 7 kDa. This banding pattern is highly indicative of polyubiquitination. To directly test if these ataxin-1 immunoreactive bands were ubiquitin conjugates, ataxin-1 [92Q] transfected cells were incubated with or without β-lactone, lysed, and the detergent soluble and insoluble fractions were subject to denaturing 6xHis-ataxin-1 pull-down. Using ataxin-1 antisera, immunoblot analysis of the affinity purified proteins from the detergent soluble fractions revealed no high molecular weight smear. By contrast, strong immunoreactivty was observed as a smear of high molecular weight material in the lane representing the affinity purified ataxin-1 from the detergent-insoluble fraction. Stripping and reprobing the same blot with anti-ubiquitin confirms that these high molecular weight forms of ataxin-1 are ubiquitin conjugates. It appears the ubiquitin immunoreactivity is present uniquely in the detergent-insoluble fractions.

### EXAMPLE 14

### SCA1 Transgenic Mice Lacking Expression of Ube3a Have Reduced NI

To evaluate the possible role of the ubiquitin pathway in SCA1, B05 mice were crossbred with the well characterized animals lacking expression of Ube3a. These experiments took advantage of the imprinted expression pattern for Ube3a resulting in preferential expression of the maternal allele in Purkinje cells and set up matings to yield SCA1 mice with a maternal deficiency for Ube3a^{(π-/p+)}. Male heterozygous B05 transgenic mice were mated with female heterozygous Ube3a mice which produced litters with the expected ratios for each genotype. As anticipated the B05, Ube3a and B05/Ube3a mice developed normally, and were indistinguishable from each other and wild-type littermates by cage behavior for the first 3 months.

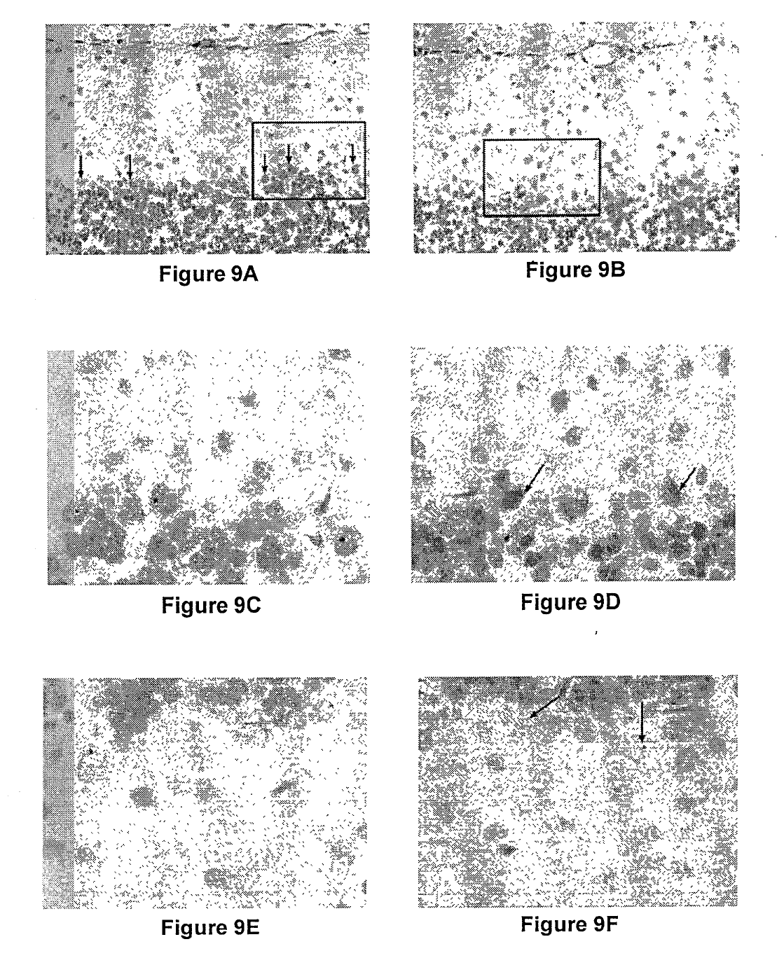

The mechanism involved in NI formation and the role the inclusions play in SCA1 pathogenesis is unclear. The NI in SCA1 B05 transgenic mice are first evident at 3.5 weeks by immunohistochemistry using either anti-ataxin-1 or anti-ubiquitin antisera. The fraction of Purkinje cells with NI increases with age until 12 weeks when they are present in 90% of these neurons. To assess the role of E6-AP in NI formation the subcellular localization of ataxin-1 in cerebellar sections from the Ube3a, B05 and B05/Ube3a mice at 6.5, 9.5, and 12.5 weeks were analyzed by immunohistochemistry. The distribution of endogenous ataxin-1 in the Ube3a^{(m-/p+)}, mice cerebellum was nearly identical to wild-type. In Purkinje cells from the transgenic mice expressing mutant ataxin-1 with no deficiency in Ube3a^{(m+/p+)}, ataxin-1 localized throughout the nucleus and to a single nuclear structure. The frequency of NI in the Ube3a^{(m+/p+)}/SCA1 B05 Purkinje cells increased with age from 38% at 6.5 weeks to Å 90% at 12.5 weeks. In contrast, the Purkinje cells in transgenic animals lacking expression of Ube3a^{(m-/p+)} had predominantly a diffuse nuclear distribution for ataxin-1 with a small number of nuclei containing micropunctate structures or a single NI. The Ube3a^{(m-/p+)}/SCA1 B05 mice had nearly a ten fold reduction in percentage of NI at 6.5 and 9.5 weeks compared to SCA1 B05 littermates. It is intriguing to note that the NI percentage increased with time as there was only a three fold difference in NI percentage at 12.5 weeks. These data indicate that the lack of this E3 ubiquitin ligase causes a delay in the appearance of NIs but that other factors are contributing to their formation. Immunohistochemical analysis using antibody to ubiquitin does demonstrate that the NI which eventually form in the Purkinje cells in the double mutant animals do contain ubiquitinated material (FIGS. 9A through 9F). Northern blot analysis demonstrated no change in expression of the SCA1 transgene in the B05/Ube3a^{(m-/p+)} animals confirming the decreased frequency of the NI formation was not due to alterations in transgene expression.

### EXAMPLE 15

### SCA1 Transgenic Mice Lacking Expression of Ube3a Have Severe SCA1 Pathology

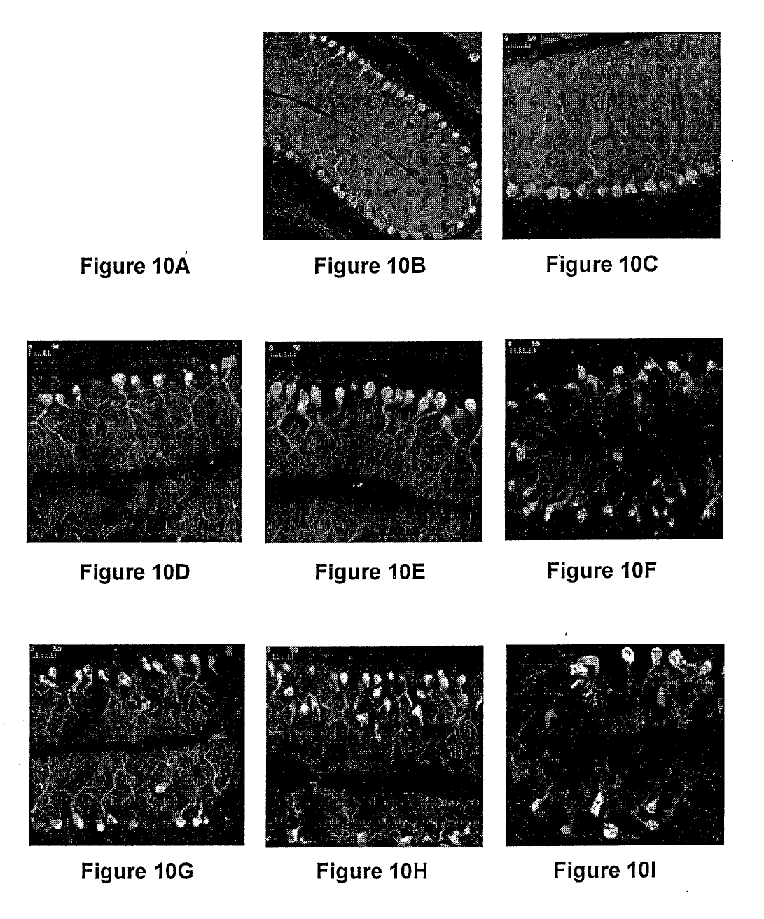

Because the frequency of the nuclear inclusion formation was reduced in the double mutant mice, it was of particular interest to compare cerebellar sections from the B05 SCA1 transgenic mice with and without expression of Ube3a to determine if any of the cellular changes observed in the B05 SCA1 mice were altered. Histopathologically, the B05/Ube3a^{(m-/p+)} cerebellum had thinning of the molecular layer, Purkinje cell vacuolation and cell bodies displaced from the Purkinje cell layer (FIGS. 10A through 10I). To ascertain a better view of the subcellular localization of ataxin-1 and dendritic morphology of the Purkinje cells, sections from animals at 9.5, 12.5 and 14.5 weeks were examined using antibodies to ataxin-1 (11NQ) and the Purkinje cell-specific protein calbindin. As was observed by light microscopy, immunofluorescence analysis with the 11NQ antibody confirmed a clear reduction in the appearance of NI in the double mutant animals. While the subcellular localization of ataxin-1 was primarily nuclear and concentrated to the NI in the B05 mice, the distribution of ataxin-1 was much more diffuse in the nucleus with limited staining in the cytoplasm in the double mutant animals. More striking however is the radical loss of dendritic aborization, vacuolation, and severe Purkinje cell heterotopia in the sections from the B05 SCA1 transgenic mice lacking Ube3a^{(m-/p+)} expression. The striking alterations in Purkinje cell morphology that develop in the double mutant mice at 14.5 weeks are comparable to that of B05 SCA1 mice at ages greater than 9 months. These results indicate that lack of expression of an E3 ubiquitin ligase accelerates the polyglutamine-induced pathology in the SCA1 transgenic animals. Additionally, this dramatic pathology is not dependent on NI formation.

### EXAMPLE 16

### Ubiquitin Pathway Involvement In NI Formation

Although the steps leading to NI formation are not completely clear, several observations indicate the ubiquitin pathway is involved. First, ubiquitin is one of the first epitopes to be recognized in the developing NI in SCA1 transgenic mice. Second, the ubiquitinated forms of ataxin-1, in HeLa cells, are uniquely found in the detergent-insoluble fraction. Third, preventing turnover of the ubiquitinated forms of ataxin-1 with proteasome inhibitors leads to increased ataxin-1 aggregation. Fourth, the frequency of NI in SCA1 transgenic animals is diminished by the absence of an E3 ubiquitin ligase.

### EXAMPLE 17

### Effect of Inhibition of Proteasome

That inhibition of the proteasome led to an increase in size and frequency of aggregates in HeLa cells indirectly indicates the proteasome is a cellular mechanism to suppress NI formation. Similarly it is known in the act that proteasome inhibition resulted in an increase in aggregate formation of truncated ataxin-3. These findings suggest the role of the proteasome in the neurodegeneration is not limited to SCA1 and may in fact extend to other neurodegenerative disease.

The highly selective and specific nature of protein degradation is in part governed by the E3 ubiquitin ligase. Given its unique expression pattern in the hippocampus and cerebellar Purkinje cells it is conceivable that E6-AP is involved the SCA1 tissue specific phenotype. Patients with a maternal deficiency in Ube3a have ataxia indicating Purkinje cell dysfunction.

### EXAMPLE 18

### Effect of E6-AP

The polyglutamine induced pathology in the SCA1 transgenic mice is characterized by cytoplasmic vacuoles, progressive loss of dendritic arborization, and Purkinje cell heterotopia. This combination of cytoplasmic vacuoles and Purkinje cell heterotopia are unique and have not been described for any other mouse mutant, neither genetic nor acquired. Thorough histopathological examination of Ube3a deficient mice revealed normal histology of the brain. It is therefore concluded that the severe, progressive pathological changes in the SCA1 transgenic mice lacking expression of Ube3a is caused by the toxic effect of mutant ataxin-1 aggravated by the lack of E6-AP function. Moreover, the severe pathology is very similar to that seen in the cerebellum of late-stage SCA1 transgenic animals suggesting the lack of E6-AP accelerates the polyglutamine-induced phenotype.

This accelerated phenotype is due to the loss of function of E6-AP which directly results in altered ataxin-1 ubiquitination and hydrolysis. If the mutant protein is not being properly tagged and degraded it is toxic because of changes in its steady-state levels. It is proposed that the toxic gain of function mechanism in SCA1 is a result of a gain of more of the normal function of ataxin-1. Detailed analysis of gene expression in the SCA1 B05 line has revealed very early changes involving a number of Purkinje cell specific genes. Altered steady state levels of ataxin-1 could conceivable yield such specific changes in gene expression.

### EXAMPLE 19

### Summary

The examples clearly indicate for the first time that molecular chaperones are involved in a glutamine repeat disease. Affected neurons from the brain stem of an SCA1 patient and Purkinje cells from transgenic mice expressing mutant ataxin-1 have ubiquitin-positive nuclear inclusions which contain the proteasome and the molecular chaperone HDJ-2/HSDJ. It appears that normally cells target HDJ-2/HSDJ to the nuclear inclusion in an ultimately unsuccessful attempt to maintain the proteins in a conformation which promotes either their refolding or their modification by the ubiquitinating enzymes and subsequent hydrolysis by the 26S proteasome. The present invention demonstrates that by overexpressing the chaperone in cultured cells, it is possible to augment cellular response to the presence of misfolded proteins. This augmented approach curbs the formation of these nuclear aggregates.

The chaperone's dual roles in aggregate formation and suppression may not be mutually exclusive, but rather dependent on the presence and level of chaperone expression. A similar phenomenon may occur in SCA1, with endogenous levels of HDJ2/HSDJ and/or Hsc70 contributing to the formation of ataxin-1 aggregates when the number of glutamine repeats is in the disease-causing range.

The observation that the J-domain mutants of HDJ-2/HSDJ were unable to suppress aggregation of ataxin-1 indicates the J-domain is necessary to prevent nuclear protein aggregation.

Hsp70 may be upregulated in HeLa cells containing large nuclear ataxin-1 aggregates, suggesting these cells are responding to an adverse change in their normal cellular environment. The actual stress signal that could causes the cell to upregulate this hsp is not clear, but it is known that agents which block proteasome function cause an accumulation of abnormal proteins and increase hsp expression¹⁶⁻¹⁸. Thus, the nuclear aggregates cause a redistribution of the proteasome and saturate the cells' degradative machinery, leading to both a failure to degrade critical short-lived proteins and a secondary upregulation of inducible hsps.

That Hsp70 is not upregulated in affected neurons in either the SCA1 patient or transgenic animals could be relevant to the nuclear aggregation and/or pathogenesis seen in these cells. Hsp70 is not usually expressed in neurons under normal conditions, but it is expressed at high levels in stressed cells^{38, 39}. This suggests that neurons affected in SCA1 are not mobilizing components of the stress response required to increase expression of Hsp70. HDJ-2/HSDJ may associate with ataxin-1 aggregates in the absence of Hsp70, but it may not be capable of suppressing aggregate formation on its own.

Purkinje cells in the transgenic mice expressing mutant ataxin-1 accumulate nuclear aggregates, probably because the cells cannot effectively process high levels of mutant protein. In the B05 line mutant ataxin-1 is expressed at over twenty times the endogenous level using Purkinje cell-specific promoter. Transgenic mice expressing mutant ataxin-1 containing 82 glutamines under the neuron-specific enolase (NSE) promoter [NSE 82Q] (equivalent to endogenous levels) never develop nuclear inclusions or a phenotype, suggesting that the refolding or proteolysis systems in the neurons of these animals are not compromised. Time, however, is likely a critical parameter in the formation of the insoluble aggregates. The life span of the NSE transgenic mice may not be sufficient to allow study of the accumulation of protein folding errors and subsequent aggregate formation. In SCA1 patients, where mutant ataxin-1 is expressed at endogenous levels, the disease is clearly progressive and the age of onset is typically on the order of decades. The inverse relationship between size of CAG repeat and age of onset is consistent with the notion that a) the long glutamine tract destabilizes the protein conformation, and b) protein-misfolding errors are likely to accumulate faster in neurons expressing a protein with a more destabilized conformation due to a longer glutamine tract.

We demonstrate that ataxin-1 aggregation is suppressed following overexpression of HDJ-2/HSDJ in HeLa cells. It has been postulated that the DnaJ and DnaK family members act together to inhibit premature protein folding and aggregation, thereby increasing the likelihood of correct protein folding. It is possible that in HeLa cells overexpressing HDJ-2/HSDJ, the recombinant chaperone protein is enhancing endogenous Hsp70 activity, thus preventing the aggregation of mutant ataxin-1. Alternatively, at elevated levels HDJ-2/HSDJ may act alone as a molecular chaperone to prevent ataxin-1 aggregation. Purified Ydjl acts as a chaperone in the absence of other proteins^{20, 40}. But overexpression of HDJ-2/HSDJ in HeLa does not prevent aggregate formation completely, perhaps because of the variability inherent in transient transfection experiments with regards to episomal copy number and relative expression levels of HDJ-2/HSDJ and ataxin-1. Other limiting factors may include DnaK or other chaperones. The Hsp90 chaperone, for example, has been found to stimulate protein renaturation brought about by Hsp70 and Ydj-1 in vitro⁴¹. Overexpression of Hsp70, related DnaK family members, or other chaperones may be necessary to prevent ataxin-1 aggregation entirely through molecular chaperone-mediated refolding.

### REFERENCES

All patents and publications mentioned in the specification are indicative of the levels of those skilled in the art to which the invention pertains. All patents and publications are herein incorporated by reference to the same extent as if each individual publication was specifically and individually indicated to be incorporated by reference.

1. Wisniewski, T., Ghiso, J. & Frangione, B. Biology of A beta amyloid in Alzheimer's disease. Neurobiol Dis 4, 313-328 (1997).

2. Polymeropoulos, M. H. et al. Mutation in the α-Synuclein Gene Identified in Families with Parkinson's Disease. Science 276(1997).

3. Prusiner, S. B. Prion diseases and the BSE crisis. Science 278, 245-251 (1997).

4. Davies, S. W. et al. Formation of neuronal intranuclear inclusions underlies the neurological dysfunction in mice transgenic for the HD mutation. Cell 90, 537-548 (1997).

5. DiFiglia, M. et al. Aggregation of Huntingtin in neuronal intranuclear inclusions and dystrophic neurites in brain. Science 277, 1990-1993 (1997).

6. Igarashi, S. et al. Suppression of aggregate formation and apoptosis by transglutaminase inhibitors in cells expressing truncated DRPLA protein with an expanded polyglutamine stretch. Nat Genet 18, 111-117 (1998).

7. Skinner, P. J. et al. Ataxin-1 with extra glutamines induces alterations in nuclear matrix-associated structures. Nature 389, 971-974 (1997).

8. Paulson, H. L. et al. Intranuclear inclusions of expanded polyglutamine protein in spinocerebellar ataxia Type 3. Neuron 19, 333-334 (1997).

9. Zoghbi, H. Y. & Orr, H. T. Spinocerebellar ataxia type 1. Seminars in Cell Biology: Unstable Repeat Diseases 6, 29-35 (1995).

10. Perutz, M. F., Johnson, T., Suzuki, M. & Finch, J. T. Glutamine repeats as polar zippers: their possible role in inherited neurodegenerative diseases. PNAS 91, 5355-5358 (1994).

11. Stott, K., Blackburn, J. M., Butler, P. J. G. & Perutz, M. Incorporation of glutamine repeats makes protein oligomerize: Implications for neurodegenerative diseases. PNAS 92, 6509-6513 (1995).

12. Hershko, A. & Ciechanover, A. The ubiquitin system for protein degradation. Annu Rev Biochem 61, 761-807 (1992).

13. Hochstrasser, M. Ubiquitin-dependent protein degradation. Annu. Rev. Genet. 30, 405-439 (1996).

14. Adams, G. M. et al. Structural and Functional Effects of PA700 and Modular Protein on Proteasomes. Journal of Molecular Biology 273, 646-657 (1997).

15. Coux, O., Tanaka, K. & Goldberg, A. L. Structure and functions of the 20S and 26S proteasomes. Annu Rev Biochem 65, 801-847 (1996).

16. Bush, K. T., Goldberg, A. L. & Nigam, S. K. Proteasome Inhibition Leads to a Heat-shock Response, Induction of Endoplasmic Reticulum Chaperones, and Thermotolerance. The Journal of Biological Chemistry 272, No. 14, 9086-9092 (1997).

17. Zhou, M., Wu, X. & Ginsberg, H. N. Evidence that a rapidly turning over protein, normally degraded by proteasomes, regulates hsp72 gene transcription in HepG2 cells. J. Biol. Chem. 271, 247-269 (1996).

18. Lee, D. H. & Goldberg, A. L. Proteasome Inhibitors Cause Induction of Heat Shock Proteins and Trehalose, Which Together Confer Thermotolerance in Saccharomyces cerevisiae. Molecular and Cellular Biology 18, 30-38 (1998).

19. Bukau, B. & Horwich, A. L. The Hsp70 and Hsp60 Chaperone Machines. Cell 92, 351-366 (1998).

20. Lu, Z. & Cyr, D. M. The Conserved Carboxyl Terminus and Zinc Finger-Like Domain of the Co-chaperone Ydjl Assist Hsp70 in Protein Folding. The Journal of Biological Chemistry 273, No. 10, 5970-5978 (1998).

21. Hartl, F. U. Molecular chaperones in cellular protein folding. Nature 381, 571-580 (1996).

22. Hendricks, J. P. & Hartl, F.-U. Molecular chaperone functions of heat shock proteins. Annu. Rev. Biochem. 62, 349-384 (1993).

23. Sherman, M. Y. & Goldberg, A. L. Involvement of chaperonin dnaK in the rapid degradation of a mutant protein in Escherichia coli. EMBO J. 11, 71-77 (1992).

24. Straus, D. B., Walter, W. A. & Gross, C. A. Escherichia coli heat shock gene mutants are defective in proteolysis. Genes Dev. 2, 1851-1858 (1988).

25. Johnson, E. S., Bartel, B., Seufert, W. & Varshavsky, V. Ubiquitin as a degradation signal. EMBO J. 11, 497-505 (1992).

26. Lee, D. H., Sherman, M. Y. & Goldberg, A. L. Involvement of the Molecular Chaperone Ydjl in the Ubiquitin-Dependent Degradation of Short-Lived and Abnormal Proteins in Saccharomyces cerevisiae. Molecular and Cellular Biology 16, No. 9, 4773-4781 (1996).

27. Burright, E. N. et al. SCA1 transgenic mice: a model for neurodegeneration caused by an expanded CAG trinucleotide repeat. Cell 82, 937-948 (1995).

28. Attaix, D. et al. Expression of subunits of the 19S complex and of the PA28 activator in rat skeletal muscle. Mol Biol Rep 24, 95-98 (1997).

29. Chellaiah, A., Davis, A. & Mohanakumar, T. Cloning of a unique human homologue of the Escherichia coli DANJ heat shock protein. Biochemica et Biophysica Acta 1174, 111-113 (1993).

30. Oh, S., Iwahori, A. & Kato, S. Human cDNA encoding DnaJ protein homologue. Biochim Biophys Acta 1174, 114-116 (1993).

31. Cyr, D. M., Lu, X. & Douglas, M. G. Regulation of Hsp70 function by a eukaryotic DnaJ homolog. J Biol Chem 267, 20927-20931 (1992).

32. Tang, Y., Ramakrishnan, C., Thomas, J. & DeFranco, D. B. A Role for HDJ-2/HSDJ in Correcting Subnuclear Trafficking, Transactivation, and Transrepression Defects of a Glucocorticoid Receptor Zinc Finger Mutant. Molecular Biology of the Cell 8, 795-809 (1997).

33. Schirmer, E. C. & Lindquist, S. Interactions of the chaperone Hsp104 with yeast Sup35 and mammalian PrP. Proc. Natl. Acad. Sci. 94, 13932-13937 (1997).

34. DebBurman, S. K., Raymond, G. J., Caughey, B. & Lindquist, S. Chaperone-supervised conversion of prion protein to its protease-resistant form. Proceedings of the National Academy of Science 94, 13938-13943 (1997).

35. Welch, W. J. & Gambetti, P. Chaperoning Brain Diseases. Nature 392, 23-24 (1998).

36. Chernoff, Y. O., Lindquist, S. L., Ono, B., Inge-Vechtomov, S. G. & Liebman, S. W. Role of the Chaperone Protein Hsp104 in Propagatoin of the Yeast Prion-Like Factor [psi+]. Science 268, 880-883 (1995).

37. Cyr, D. M., Langer, T. & Douglas, M. G. DnaJ-like proteins: molecular chaperones and specific regulators of Hsp70. TIBS 19, 176-181 (1994).

38. Dienel, G., Kiessling, M., Soubrie, P., Bockaert, J. & Pin, J. Synthesis of heat shock proteins in rat brain cortex after transient ischemia. J. Cereb. Blood Flow Metab. 6, 505-510 (1986).

39. Brown, I. R. Induction of heat shock (stress) genes in the mammalian brain by hyperthermia and other traumatic events: a current perspective. J. Neurosci. Res. 27, 247-255 (1990).

40. Cyr, D. M. Cooperation of the molecular chaperone Ydjl with specific Hsp70 homologs to suppress protein aggregation. FEBS 359, 129-132 (1995).

41. Schumacher, R. J. et al. Cooperative action of Hsp70, Hsp90, and DnaJ proteins in protein renaturation. Biochemistry 35, 14889-14898 (1996).

42. Matilla, T. et al. The cerebellar leucine rich acidic nuclear protein interacts with ataxin-1. Nature 389, 974-978 (1997).

43. Shibatani, T. & Ward, W. F. Sodium dodecyl sulfate (SDS) activation of the 20S proteasome in rat liver. Arch Biochem Biophys 321, 160-166 (1995).

44. Servadio, A. et al. Expression analysis of the ataxin-1 protein in tissues from normal and spinocerebellar ataxia type 1 individuals. Nature Genetics 10, 94-98 (1995).

44. Remington's Pharmaceutical Sciences.

One skilled in the art readily appreciates that the invention is well adapted to carry out the objectives and obtain the ends and advantages mentioned as well as those inherent therein. The methods of treating neurological disease with chaperones and chaperone-like-compounds, the methods of screening for chaperone activity, compounds, pharmaceutical compositions, treatments, methods, procedures and techniques described herein are presently representative of the preferred embodiments and are intended to be exemplary and are not intended as limitations of the scope. Changes therein and other uses will occur those skilled in the art which are encompassed within the spirit of the invention or defined by the scope of the pending claims.

What we claim is:

1. A method of treating neurodegenerative disease in a mammal comprising the steps of introducing a therapeutic effective amount of a chaperone or chaperone-like-compound into the neurological system of the mammal.

2. The method of claim 1, wherein the introducing step includes introducing the chaperone or chaperone-like-compound into the mammal by gene therapy.

3. The method of claim 1, wherein the introducing step includes directly injecting the chaperone or chaperone-like-compound into the mammal.

4. A method for screening for a test compound for chaperone-like activity for the treatment of neurodegenerative diseases comprising the steps of:
introducing the test compound into transfected cells in tissue culture, wherein such transfected cells produce protein aggregate; and
measuring the quantity of protein aggregate, wherein a test compound which decreases the quantity of protein aggregate as compared to control cells has chaperone activity.

5. A method for screening for a test compound for chaperone-like activity for the treatment of neurodegenerative diseases comprising the steps of:
introducing the test compound into an animal which models neurodegenerative disease;
allowing said animal to develop; and
subsequently measuring the quantity of aggregates in said animal wherein decreased aggregate formation over control animals indicates chaperone-like activity.

6. A method of treating neurodegenerative disease in a mammal comprising the step of introducing a therapeutically effective amount of a compound into said mammal wherein said compound increases the effective concentration of a chaperone in the neurological system.

7. A method of treating neurodegenerative disease in a mammal comprising the step of introducing a therapeutically effective amount of a compound into said mammal wherein said compound increases the effective concentration or enhances the activity of a proteasome in the neurological system.

8. A method for screening for a test compound which increases proteasome activity for the treatment of neurodegenerative diseases comprising the steps of:
introducing the test compound into transfected cells in tissue culture, wherein such transfected cells produce protein aggregate; and
measuring the quantity of protein aggregate, wherein a test compound which decreases the quantity of protein aggregate is selected.

9. A method for screening for a test compound which increases proteasome activity for the treatment of neurodegenerative diseases comprising the steps of:
introducing the test compound into an animal which models neurodegenerative disease;
allowing said animal to develop; and
subsequently measuring the quantity of aggregates in said animal wherein a compound which shows decreased aggregate formation over control animals is selected.

10. Transgenic mice capable of overexpression of HDJ-2.
